(19) United States
(12) Patent Application Publication
(10) Pub. No.: US 2003/0124127 A1
(43) Pub. Date: Jul. 3, 2003

(54) Targeting leukemia cells

(76) Inventor: Lijun Yang, Gainesville, FL (US)

Correspondence Address: Stanley A. Kim, Ph. D., Esq., Akerman, Senterfitt & Eidson, P.A., 222 Lakeview Avenue, Suite 400, P.O. Box 3188, West Palm Beach, FL, 33402-3188, US

(21) Appl. No.: 10/313,250

(22) Filed: Dec. 6, 2002

Related U.S. Application Data
(60) Provisional application No. 60/338,373, filed on Dec. 6, 2001.

(51) Int. Cl. A61K 39/395; A61K 31/203; A61K 31/07

(52) U.S. Cl. 424/146.1; 424/178.1; 514/569; 514/725

(57) ABSTRACT

A molecule is targeted to a leukemia cell by first contacting the cell with a retinoid in an amount effective to increase the expression of a marker in the cell, and the contacting the cell with an agent that specifically binds the marker. For directing a molecule to an acute promyelocytic leukemia cell, the cell is contacted with all-trans retinoic acid to induce or increase expression of CD52 on the cell. The cell is then contacted with a molecule, such as an anti-CD52 antibody, that specifically binds the CD52 expressed on the cell.

### CROSS REFERENCE TO RELATED APPLICATIONS

This application claims priority from U.S. provisional patent application serial No. 60/338,373, filed Dec. 6, 2001.

### FIELD OF THE INVENTION

The invention relates to the fields of medicine, immunology and oncology. More particularly, the invention relates to an improved method for treating leukemia.

### BACKGROUND OF THE INVENTION

Acute promyelocytic leukemia (APL) is a rapidly progressive cancer of the blood that is characterized by the uncontrolled proliferation of an immature form of a white blood cell termed a promyelocyte. Overproduction of these cells crowds the bone marrow, radically reducing the body's ability to form other normal and necessary blood cells. APL is a lethal leukemia and accounts for more than 10% of all acute myeloid leukemia cases (Powell B L, Curr. Opin. Oncol. 13:8-13, 2001). The disease is characterized by distinctive and unique clinico-pathologic features including: (1) the accumulation in the bone marrow of tumor cells blocked at the promyelocytic stage of myeloid differentiation, (2) translocations involving chromosomes 15 and 17, (3) sensitivity of APL blasts to the differentiating action of retinoic acid, (4) young age of onset, (5) frequent presentation with low white blood cell counts, and (6) severe consumptive coagulopathy with a high incidence of early fatal hemorrhages (Falanga A et al., Acta Hematol. 106:43-51, 2001). Without treatment, an APL patient's life expectancy is approximately a month or even less.

Interest in APL has been stimulated by the discovery that a gene encoding the protein for retinoic acid (RA) receptor-α (RAR-α) was reciprocally translocated to a locus called promyelocytic leukemia (PML), resulting in formation of the chimeric PML-RARα oncogene (Rowley et al., Lancet 1: 549-550, 1977; Kakizuka A et al., Cell 66: 663-674, 1991; Mann K K et al., Curr. Oncol. Rep. 3: 209-216, 2001). APL blast cells are known to display exquisite sensitivity to all-trans-retinoic acid (ATRA), a physiologically active derivative of vitamin A. Upon ATRA stimulation, these cells undergo granulocytic differentiation (Breitman T R et al., Blood 57: 1000-1004, 1981). ATRA has been shown to induce differentiation of APL cells by acting directly on the PML-RARα fusion protein (Benoit G et APL have been facilitated by availability of cell lines derived from APL patients, such as NB4, a maturation inducible cell line carrying the characteristic t (15;17) translocation and expressing both the PML-RARα mRNA transcript and the fusion protein (Lanotte M et al., Blood 77:1080-1086, 1991).

The effect of ATRA on APL cells has been exploited to develop a treatment for patients suffering from this leukemia (Frankel S R et al., Ann. Intern. Med. 120: 278-286, 1994). This treatment, termed “APL differentiation therapy,” stimulates differentiation of the abnormal promyelocytes, permitting the bone marrow to replace these abnormal cells with normal white cells (Andreef M et al., Blood 80: 2604-2609, 1992). For newly diagnosed APL, ATRA therapy can result in complete remission in up to 85% of patients. Unfortunately, this remission is often short-lived.

### SUMMARY OF THE INVENTION

What has been discovered is that ATRA modulates expression of several different genes in leukemia cells. These genes and their products can thus serve as targets for delivering various agents to the leukemia cells. In the examples described below, ATRA treatment of APL cells caused increased expression of SH3-binding domain glutamic acid rich like protein, 2′,5′-oligoadenylate synthetase 1, EST highly similar to CGI-117 protein (H. Sapiens), integrin alpha 2b, homeobox B7, T-cell immune regulator 1, dual specificity phosphatase 6, M100HG, PA28 beta, novel human gene mapping to chromosome 13, N-acetyltransferase 2, Ca²⁺-dependent activator protein for secretion, calpain small polypeptide, KIAA0876 protein, G antigen 7, cytochrome P450, EST highly similar to CG1-122 protein (H. Sapiens), KIAA0545 protein, sodium-coupled nucleoside transporter, myo-inositol-1-phosphate synthase, CD52, v-myb avian myeloblastosis viral oncogene homolog-like 2, proenkephalin, peptidyl arginine deiminase type II, proteinase 3, cytokeratin type II, ICAM1, defensin, EST gf204_{—}266B9, EST gf204_{—}250G11, EST gf205_{—}305C5, EST gf205_{—}273F1, EST gf205_{—}274A10, EST gf205_{—}287A3, EST gf205_{—}278G4, EST gf205_{—}276A10, EST gf206_{—}341G4, EST gf204_{—}251A2, and EST gf206_{—}339D4. Based on this discovery, substances such as cytotoxins and detectable labels can be delivered to the ATRA-treated cells using an agent that specifically binds one or more of the products of these upregulated genes.

Of the foregoing markers, CD52 is particularly attractive for a number of different reasons. CD52 is a 21 to 28-kd non-modulating cell surface glycosyl-phosphatidyl-inositol-linked glycoprotein of unknown function (Xia M Q et al., Biochem.J. 293 Pt 3: 633-640, 1993). Previous studies have shown it to be abundantly expressed on most normal and malignant T and B-lymphocytes (Hale G et al., Tissue Antigens 35: 118-127, 1990), monocytes (Xia M Q et al., Eur. J. Immunol. 21: 1677-1684, 1991), macrophages (Gilleece M H et al., Blood 82: 807-812, 1993) and cosinophils but not neutrophils (Elsner J et al., Blood 88: 4684-4693, 1996).

CD52 is also known to be an excellent target for complement-mediated cell lysis and antibody-dependent cell-mediated cytotoxicity (Dyer M J et al., Blood 73: 1431-1439, 1989). A rat monoclonal antibody (CAMPATH-1) that recognizes CD52 is known to be unusually effective at causing cell lysis with homologous complement. The CD52 antigen recognized by this antibody has been shown to be a very small GPI-anchored glycoprotein with a mature peptide comprising only 12 amino acids. The efficacy of cell lysis is thought to be due to the presence of the CAMPATH-1 binding epitope very close to the lipid bilayer (Xia M Q et al., Biochem. J. 293 Pt. 3: 633-640, 1993).

A humanized anti-CD52 monoclonal antibody (CAMPATH-1H) has been used clinically to treat a number of lymphoproliferative disorders (Hale G et al., Lancet 2: 1394-1399, 1988; Dyer M J et al., Blood 73: 1431-1439, 1989; Dearden C E et al., Blood 98: 1721-1726, 2001) and to delete lymphocytes from organ transplants (Naparstek E et al., Exp.Hematol. 27: 1210-1218, 1999). Notably, however, CAMPATH-1 has been found to have no effect on myeloid leukemia, presumably because myeloid cells are known to express little, if any, CD52 (Domagala A and Kurpitz, M, Med. Sci. Monit. 7: 325-331, 2001). The CAMPATH-1 antibody also has no effect on the biological function of normal neutrophils, which likewise do not express CD52 (Fabian I et al., Exp. Hematol. 21: 1522-1527, 1993).

Accordingly, the invention features a method for targeting a molecule to a leukemia cell. The method includes the steps of: (a) first contacting the cell with a retinoid in an amount effective to increase the expression of a marker in the cell; and (b) second contacting the cell with an agent that specifically binds the marker. The agent can be the molecule itself, a substance to which the molecule is attached, or any other substance that specifically binds the marker. The marker can be any gene product upregulated in a leukemia cell by ATRA. In a particular embodiment of the method, the marker on the leukemia cell is CD52 and the agent is an anti-CD52 antibody.

In another aspect, the invention also provides a kit for targeting a molecule to a leukemia cell. The kit includes (a) a retinoid (e.g., ATRA) for increasing the expression of a marker (e.g., CD52) in the cell; and (b) an agent that specifically binds the marker.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including any definitions will control. In addition, the particular embodiments discussed below are illustrative only and not intended to be limiting.

By the term “retinoid” is meant any molecule that binds a retinoic acid receptor.

As used herein, a “marker” is any molecule whose expression in a cell can be modulated in response to treatment with a retinoid. Examples of such markers include nucleic acids and polypeptides.

The term “cytotoxin” as used herein means a molecule or compound that is destructive to living cells. Examples of cytotoxins include radionuclides, conjugated radionuclides, and chemotherapeutic agents.

By the phrase “specifically binds” is meant that one molecule in a mixture recognizes and adheres to a particular second molecule in the mixture, but does not substantially recognize or adhere to other molecules dissimilar from the second molecule in the mixture. Generally, a first molecule that “specifically binds” a second molecule has a binding affinity greater than about 10⁵ to 10⁶ liters/mole for that second molecule.

By the term “antibody” is meant an immunoglobulin as well as any portion or fragment of an immunoglobulin whether made by enzymatic digestion of intact immunoglobulin or by techniques in molecular biology. The term also refers to a mixture containing an immunoglobulin (or portion or fragment thereof) such as an antiserum.

### BRIEF DESCRIPTION OF THE DRAWINGS

The above and further advantages of this invention may be better understood by referring to the following description taken in conjunction with the accompanying drawings, in which:

FIG. 1 is a series of histograms from flow cytometric analysis of NB4 cells treated with ATRA. Cell differentiation was evaluated by CD11B and CD16 expression.

FIG. 2 is a graph depicting ATRA-induced NB4 cell differentiation. NB4 cells were treated with ATRA for 12, 24, 48, 72 and 96 hrs. Cell differentiation was evaluated by the nitroblue tetrazolium (NBT) reduction test.

FIG. 3 is a graph depicting ATRA-induced NB4 cell growth inhibition of cells treated with 1 μM ATRA for 12, 24, 48, 72 and 96 hrs. For cell cycle analysis (% of cells in S-phase), cells were stained with propidium iodide (PI) and analyzed by flow cytometry.

FIG. 4 is a hierarchical cluster analysis of genomic expression patterns in ATRA-induced NB4 cell differentiation. Tree View and Cluster Analysis Programs were employed. The exploded view in the right panel shows a partial list of ATRA-induced genes that clustered to adjacent nodes, with levels of gene expression ranging from low to high. The length of the branches on the tree view indicates the degree of difference between the gene expression patterns of the various genes. The different lanes represent different time points of incubating NB4 cells with ATRA. Control was NB4 cells in the absence of ATRA.

FIG. 5 is a graphic summarizing numbers of genes in NB4 cells showing a 2-fold change in level of expression at the indicated time points following ATRA treatment.

FIG. 6 is a graphic listing examples of 29 known genes displaying 3-fold upregulation at 96 hrs post ATRA administration. Expression changes in CDw52 antigen (CAMPATH-1 antigen) are shown in boldface.

FIG. 7 is a graphic depicting unknown genes that are at least 3-fold upregulated at 96 hrs post ATRA administration. The graph on the right shows increasing elevation in expression of gene gf206_{—}339D4 from 12 to 96 hr of ATRA treatment, reaching more than 58-fold of control at 96 hr.

FIG. 8 is an agarose gel and a Western blot showing induction of CD52 expression by ATRA in NB4 cells. (A) RT-PCR analysis of CD52 and β-actin mRNA and (B) Western blot analysis of CD52 protein induction in NB4 cells treated with ATRA at the indicated time points. No CD52 mRNA and protein were detected in NB4 cells untreated with ATRA (0 h). β-actin expression in (A) confirms similar RNA loading in each lane.

FIG. 9 is a series of histograms showing flow cytometric analysis of CD11B, CD18, and CD52 surface expression in NB4 cells after exposure to ATRA for 24, 48, 72 and 96 hours. Open and filled histograms represent untreated control and ATRA-treated cells, respectively. Kinetics of ATRA-induced CD11B, CD18, and CD52 expression are similar at each time point.

FIG. 10 shows the expression of CD11B and CD52 on leukemia cell lines U937, K562, HL-60 and NB4, treated with ATRA for 96 hours. Up-regulation of CD52 by ATRA occurs only in NB4 cells. The upper panels represent control cells that did not receive ATRA. The bottom panels represent cells that received ATRA.

### DETAILED DESCRIPTION OF THE INVENTION

The invention provides methods and compositions for specifically targeting substances to leukemia cells. In the invention, a leukemia cell is first contacted with a retinoid in an amount effective to increase expression of at least one marker in the cell. The cell is then contacted with an agent that specifically binds the marker whose expression was increased. The molecule targeted to the cell can be the agent itself or another substance that is attached or otherwise associated with the agent.

The below described preferred embodiments illustrate adaptations of these compositions and methods. Nonetheless, from the description of these embodiments, other aspects of the invention can be made and/or practiced based on the description provided below.

### Biological Methods

Methods involving conventional biology techniques are described herein. Such techniques are generally known in the art and are described in detail in methodology treatises such as Molecular Cloning: A Laboratory Manual, 3nd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

### Modulating Expression of a Marker in a Leukemia Cell

The invention utilizes a method for modulating (e.g., upregulating or downregulating) expression of one or more markers in a leukemia cell. Expression of a particular marker in a leukemia cell is modulated by contacting the cell with a retinoid in an amount effective to upregulate or downregulate expression of the marker in the cell. The leukemia cell used in the method can be any having genes encoding markers whose expression can be modulated in response to treatment with a retinoid. The leukemia cell can be one in in vitro culture or one within an animal subject (e.g., a human being). A number of different leukemia cell types are known. Those that might be used in the invention include leukemia cells of lymphoid or myeloid lineage. The invention is particularly important for targeting mycloid leukemia cells, as, heretofore, such cells have not been shown to express high levels of CD52. Examples of myeloid leukemia cells include acute myelogenous leukemia (AML) and APL cells. These and other leukemia cells are described in detail in Applebaum et al., Hematology, 62-86, 2001 and Byrne and Marshall, Br. J. Haematol., 100:256-64, 1998.

The retinoid used can be any that can modulate expression of a given marker in the cell. Examples of retinoids include retinols, retinoic acids, and retinyl esters. By the term “retinol” is meant any isomers of retinol, e.g., all-trans-retinol, 13-cis-retinol, 11-cis-retinol, 9-cis-retinol, 3,4-didehydro-retinol. Examples of retinoic acids include all trans retinoic acid (ATRA) and 9-cis retinoic acid. Examples of retinyl esters include: retinyl palmitate, retinyl formate, retinyl acetate, retinyl propionate, retinyl butyrate, retinyl valerate, retinyl isovalerate, retinyl hexanoate, retinyl heptanoate, retinyl octanoate, retinyl nonanoate, retinyl decanoate, retinyl undecandate, retinyl laurate, retinyl tridecanoate, retinyl myristate, retinyl pentadecanoate, retinyl heptadeconoate, retinyl stearate, retinyl isostearate, retinyl nonadecanoate, retinyl arachidonate, retinyl behenate, retinyl linoleate, and retinyl oleate. To the extent that they can modulate gene expression in a leukemia cell other synthetically prepared retinoids might be used in the invention; see e.g., U.S. Pat. Nos. 4,326,055 and 5,234,926, and 5,827,878. Whether a given retinoid is suitable for use in the invention can be determined empirically. For example, a retinoid to be tested can be added to a leukemia cell culture, and expression of CD52 or another marker on the cells can be assessed at later time points. Additionally, the retinoid to be tested can be administered to an animal, leukemia cells can be isolated from the animal, and the cells can be analyzed for expression of CD52 or another marker.

An amount of a retinoid effective to increase expression of at least one marker in the cell can also be determined empirically. For example, different concentrations of a retinoid to be tested can be added to a culture containing a leukemia cell or administered to an animal subject containing a leukemia cell. Expression of a marker on the cells can be assessed at later time points. Those concentrations that cause modulation of marker expression are concentrations suitable for use in the invention. For cultures containing a leukemia cell, suitable concentrations of ATRA are expected to range from 1-10 μmolar. For administration to an animal subject, suitable dosages will depend on many factors, including the subject's size, body surface area, age, the particular composition to be administered, time and route of administration, general health, and other drugs being administered concurrently. It is expected that an appropriate retinoid dosage for intramuscular administration of retinoids (e.g., ATRA) would be in the range of about 5.0 to 125.0 mg/square meter body surface area.

Cells in in vitro culture can be contacted with a retinoid simply by adding the retinoid to the culture. Cells in an animal subject can be contacted with a retinoid by administering the retinoid to the subject. Methods of administering retinoids to animals, including dosage regimens, are known. See, e.g., Wang et al., Chin. Med. J. 112:963-7, 1999; Kohno et al., Leuk. Lymphoma, 42:151-61, 2001; and Mann et al., Ann. Hematol. 80:417-22, 2001.

### Targeting Leukemia Cells

The invention includes a method for targeting a molecule to a leukemia cell treated with a retinoid to increase expression of a marker in the cell. A molecule is targeted to the cell by contacting the cell with an agent that specifically binds the marker. The molecule itself can be the agent that specifically binds the marker, or it can be another substance that is attached or otherwise associated with the agent.

The agent that specifically binds the marker can be anything that specifically binds to the marker. Common examples of such agents include marker-specific antibodies, naturally occurring ligands that specifically bind the marker, and artificially created agents that specifically bind the marker.

In some embodiments, the molecule targeted to the leukemia cell can be an antibody. Any antibody that specifically binds to a marker whose expression is upregulated in a leukemia cell in response to retinoid stimulation can be used. Methods for production of antibodies are well known. Antibodies for use in the invention include polyclonal antibodies and, in addition, monoclonal antibodies, single chain antibodies, Fab fragments, F(ab′)₂ fragments, and molecules produced using a Fab expression library. Monoclonal antibodies, which are homogeneous populations of antibodies to a particular antigen, can be prepared using the selected polypeptides described above and standard hybridoma technology. See e.g., Kohler et al. (1975) Nature 256:495; Kohler et al. (1976) Eur. J. Immunol. 6:511; Kohler et al. (1976) Eur. J. Immunol. 6:292; Hammerling et al. (1981) Monoclonal Antibodies and T Cell Hybridomas, Elsevier, N.Y. In particular, monoclonal antibodies can be obtained by any technique that provides for the production of antibody molecules by continuous cell lines in culture such as described in Kohler et al. (1975) Nature 256:495 and U.S. Pat. No. 4,376,110; the human B-cell hybridoma technique (Kosbor et al. 1983) Immunology Today 4:72; Cole et al. (1983) Proc. Natl. Acad. Sci. USA 80:2026), and the EBV-hybridoma technique (Cole et al. (1983) Monoclonal Antibodies and Cancer Therapy, Alan R. Liss, Inc., pp. 77-96). Such antibodies can be of any immunoglobulin class including IgG, IgM, IgE, IgA, IgD and any subclass thereof. A hybridoma producing a mAb of the invention may be cultivated in vitro or in vivo. The ability to produce high titers of mAbs in vivo makes this a particularly useful method of production.

Techniques described for the production of single chain antibodies (U.S. Pat. Nos. 4,946,778, 4,946,778, and 4,704,692) can be adapted to produce single chain antibodies against a polypeptide of interest, or a fragment thereof. Single chain antibodies are formed by linking the heavy and light chain fragments of the Fv region via an amino acid bridge, resulting in a single chain polypeptide.

Antibody fragments that recognize and bind to specific epitopes can be generated by known techniques. For example, such fragments include but are not limited to F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed (Huse et al. (1989) Science 246:1275) to allow rapid and easy identification of monoclonal Fab fragments with the desired specificity.

In one aspect of the invention, preferred antibodies are those that can directly bring about a cytotoxic effect, e.g., an anti-CD52 antibody such as the CAMPATH-1H antibody. The CAMPATH-1H antibody has emerged as a potential agent for front-line treatment of certain other leukemias. In contrast to the situation with APL cells, CD52 is abundantly expressed on many normal and malignant lymphocytes and monocytes. Clinical studies have shown CAMPATH-1H to be extremely effective at mediating depletion of both normal and malignant lymphocytes in vivo. This antibody has found use in treatment of some chronic leukemias, such as T-cell prolymphocytic leukemia and certain B-cell chronic lymphocytic leukemias, including non-Hodgkin's lymphoma and chronic lymphocytic leukemia, as well as for multiple sclerosis, and other autoimmune diseases, solid organ and bone marrow transplants, and graft versus host disease.

In other embodiments, the molecule targeted to the leukemia cell can be conjugated to a cytotoxin. A molecule conjugated with one or more cytotoxins can be used, for example, to kill cells expressing a receptor to which the molecule binds. Cytotoxins for use in the invention can be any cytotoxic agent (e.g., molecule that can kill a cell after contacting the cell) that can be conjugated to a molecule of interest, including an antibody. Examples of cytotoxins include, without limitation, radionuclides (e.g., ³⁵S, ¹⁴C, ³²P, ¹²⁵I, ¹³¹I, ⁹⁰Y, ⁸⁹Zr, ²⁰¹Tl, ¹⁸⁶Re, ¹⁸⁸Re, ⁵⁷Cu, ²¹³Bi, ²¹¹At, etc.), conjugated radionuclides, and chemotherapeutic agents. Further examples of cytotoxins include, but are not limited to, antimetabolites (e.g., 5-flourouricil (5-FU), methotrexate (MTX), fludarabine, etc.), anti-microtubule agents (e.g., vincristine, vinblastine, colchicine, taxanes (such as paclitaxel and docetaxel), etc.), alkylating agents (e.g., cyclophasphamide, melphalan, bischloroethylnitrosurea (BCNU), etc.), platinum agents (e.g., cisplatin (also termed cDDP), carboplatin, oxaliplatin, JM-216, CI-973, etc.), anthracyclines (e.g., doxorubicin, daunorubicin, etc.), antibiotic agents (e.g., mitomycin-C), topoisomerase inhibitors (e.g., etoposide, tenoposide, and camptothecins), or other cytotoxic agents such as ricin, diptheria toxin (DT), Pseudomonas exotoxin (PE) A, PE40, abrin, saporin, pokeweed viral protein, ethidium bromide, glucocorticoid, anthrax toxin and others. See, e.g., U.S. Pat. No. 5,932,188.

The molecule targeted to the leukemia cell can also be conjugated to a detectable label. Useful detectable labels in the present invention include biotin or streptavidin, magnetic beads (e.g., Dynabeads™), fluorescent dyes (e.g., fluorescein isothiocyanate, texas red, rhodamine, green fluorescent protein, and the like), radiolabels (e.g., ³H, ¹²⁵I, ³⁵S, ¹⁴C, ³²P, ¹¹¹In, ⁹⁷Ru, ⁶⁷Ga, ⁶⁸Ga, or ⁷²As), radioopaque substances such as metals for radioimaging, paramagnetic agents for magnetic resonance imaging, enzymes (e.g., horseradish peroxidase, alkaline phosphatase and others commonly used in an ELISA), and colorimetric labels such as colloidal gold or colored glass or plastic (e.g., polystyrene, polypropylene, latex, etc.) beads.

Means of detecting such labels are well known to those of skill in the art. Thus, for example, radiolabels may be detected using photographic film or scintillation counters. Fluorescent markers may also be used and can be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

### Kits

The invention also features kits for targeting a molecule to a leukemia cell. Such kits can include a container holding one or more of the retinoids for increasing expression of a marker in a cell and an agent that specifically binds the marker described above in a pharmaceutically acceptable form.

### Administration of Compositions

The compositions of the invention may be administered to animals including humans in any suitable formulation. For example, retinoids may be administered in neat form. Retinoids and cytotoxic agents may also be formulated in pharmaceutically acceptable carriers or diluents such as physiological saline or a buffered salt solution. Suitable carriers and diluents can be selected on the basis of mode and route of administration and standard pharmaceutical practice. For example, retinol is practically insoluble in water or aqueous salt solutions, but soluble in ethanol, methanol, ether, fats, and oil. For injection, retinol can be dissolved in corn oil. For addition to tissue culture, retinol can be dissolved in a suitable amount of ethanol. A description of other exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions. For example, glycine (e.g., 0.3M, pH 6.8), maltose (e.g., 10%) and/or thimerosal (e.g., 1:10,000) may be added to the compositions.

Cytotoxic agents (e.g., CAMPATH-1H antibody) may be administered in a variety of ways, including intravenously and subcutaneously. Methods of administering CAMPATH-1H, including dosage regimens, are described, for example, in Dearden et al., Blood 98: 1721-6, 2001; Pawson et al., J. Clin. Oncol. 15: 2667-72; and Bowen et al., Br. J. Haematol. 96: 617-9, 1997.

The compositions of the invention may be administered to animals by any suitable technique. Typically, such administration will be parenteral (e.g., intravenous, subcutaneous, intramuscular, or intraperitoneal introduction). The compositions may also be administered directly to a target site by, for example, surgical delivery to an internal or external target site, or by catheter to a site accessible by a blood vessel. Other methods of delivery, e.g., liposomal delivery or diffusion from a device impregnated with the composition, are known in the art. Oral delivery of the compositions might also be used in some cases. The compositions may be administered in a single bolus, multiple injections, or by continuous infusion (e.g., intravenously or by peritoneal dialysis). For parenteral administration, the compositions are preferably formulated in a sterilized pyrogen-free form.

### EXAMPLES

### Example 1

### ATRA-Induced APL Cell Differentiation

Leukemia cell lines. NB4 cells were a gift (Dr. James R. Downing, St. Jude Children's Research Hospital) and other leukemia cell lines (K562, U937 and HL-60) were purchased from ATCC (Manassas, Va.). All leukemia cell lines were cultured in RPMI 1640 medium plus 10% fetal calf serum. Cells (1×10⁶) were treated with or without 1 μM ATRA (Sigma, St. Louis, Mo.) for 12, 24, 48, 72, and 96 hours at 37° C. in a 5% CO₂ incubator in darkness. All experiments were repeated three times.

Assessment of cell differentiation. ATRA-induced NB4 cell differentiation was evaluated by morphological assessment, by flow cytometry for expression of cell surface antigens related to granulocytic differentiation and maturation such as CD11B/CD18-integrins (Gahmberg C G, Curr. Opin. Cell Biol. 9: 643-650, 1997) and by the nitroblue tetrazolium (NBT) reduction test, which measures myelocyte maturity (Idres N et al., Cancer Res. 61: 700-705, 2001). For cell cycle analysis, the cells were stained with propidium iodide (PI) and analyzed by a fluorescence-activated cell sorting analysis.

NB4 cells were analyzed for various parameters of differentiation at 12, 24, 48, 72, and 96 hrs post treatment with ATRA. No obvious morphologic change was observed in the NB4 cells at the end of 24 hrs of ATRA treatment. Morphological analysis of Giemsa stained cells showed that the ratio of cytoplasm to nucleus increased, chromatin condensation progressed gradually, and sequentially maturing myeloid cells appeared, including band forms and some segmented granulocytes. An increase in the number of apoptotic cells (as determined by morphological criteria) was noted during the time course of prolonged incubation with ATRA.

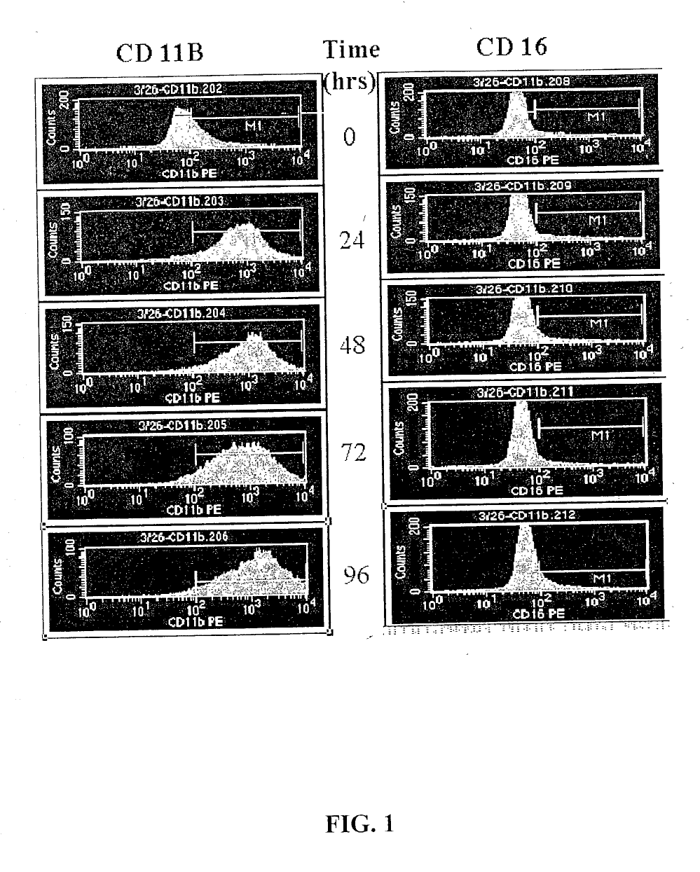

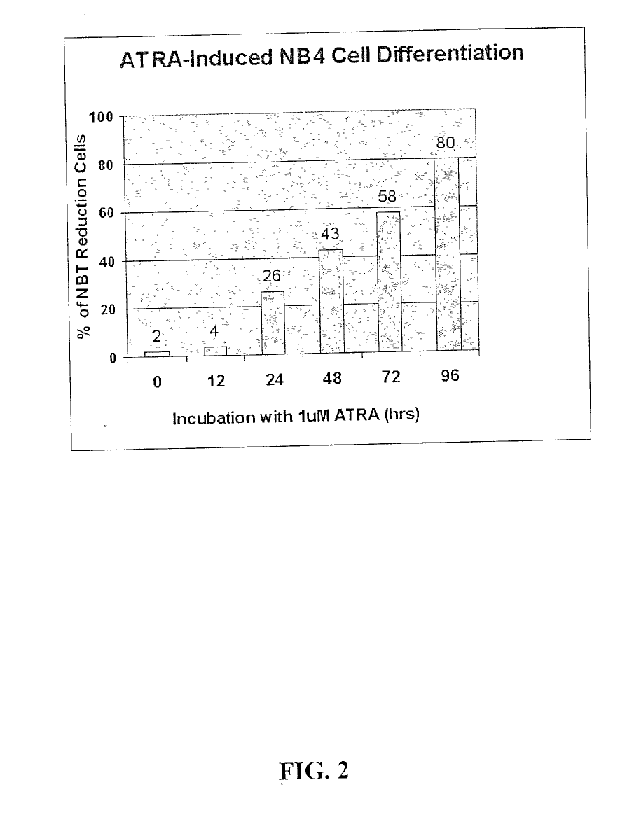

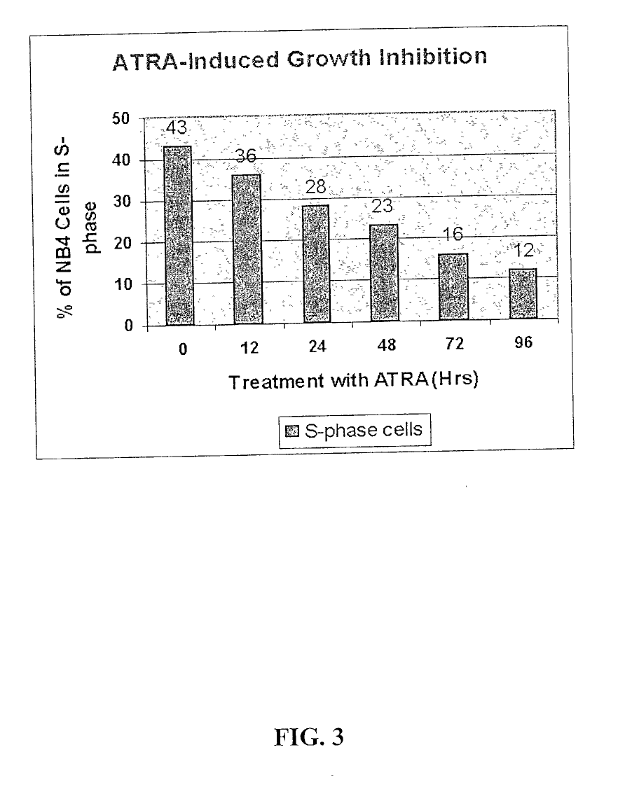

After 24 hrs ATRA treatment, differentiation markers began to be observable. CD11B+ and NBT+ cells gradually increased from 24 to 96 hrs after treatment and resulted in maximal differentiation (85%) at a later time. Flow cytometric analysis of expression of CD11B and CD16 showed that NB4 cells induced to differentiate by ATRA increasingly expressed CD11B (FIG. 1, left panel), however, they failed to express CD16, a surface marker for normal mature neutrophils (FIG. 1, right panel). Analysis by the NBT reduction test showed steadily increasing numbers of NBT+ cells starting at 24 hr (FIG. 2). Flow cytometric analysis of propidium iodide stained cells further revealed an increase of the G0/G1-phase cells and concomitant decrease of S-phase cells (FIG. 3), in parallel with the progression of cell differentiation and programmed cell death.

### Example 2

### Microarray Analysis of ATRA-Induced NB4 Cell Gene Expression

NB4 cells were cultured as described above and treated with or without 1 μM of ATRA for 12, 24, 48, 72, and 96 hrs. Total RNA was extracted using the Trizol reagent. To enhance reproducibility, RNAs from each of the indicated time points were pooled from 3 independent sets of ATRA-stimulated NB4 cells and used for the cDNA microarray studies.

Gene chips were obtained from Research Genetics. Each cDNA array chip contained 12,288 human genes, both known and unknown. The cDNA clones on the slides were sequence-verified human IMAGE clones forming part of the UniGene set. The signals were analyzed and normalized by the signal intensities of housekeeping genes. Only those genes showing signal differences of more than 2-fold over control were considered to be ATRA-modulated genes. The experimental variation of the microarray system was determined by labeling the same RNA repeatedly, followed by hybridization with multiple slides. The 95% confidence threshold ranged from 1.3-1.8 fold, suggesting that differential expression of >1.8 fold had at least a 95% likelihood of representing a true difference in gene expression. Results of hybridization analyses using RNA from control and ATRA-treated NB4 cells were analyzed by hierarchical cluster analysis of genomic expression patterns.

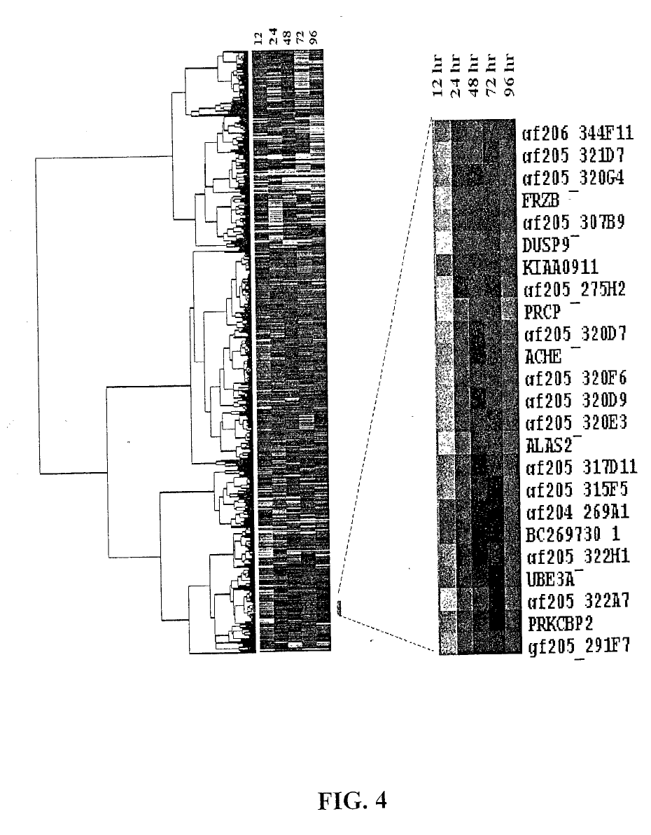

A typical hierarchical cluster analysis of genomic expression patterns in ATRA-induced NB4 cell differentiation is shown in FIG. 4. The left panel shows a visual representation of gene expression patterns using Tree View and Cluster Analysis Programs. The exploded view in the right panel shows a partial list of ATRA-induced genes that clustered to adjacent nodes, with levels of gene expression ranging from low to high. The length of the branches on the tree view indicates the degree of difference between the gene expression patterns of the various genes. The lanes marked 12, 24, 48, 72, and 96 represent different time points (in hours) of incubating NB4 cells with ATRA.

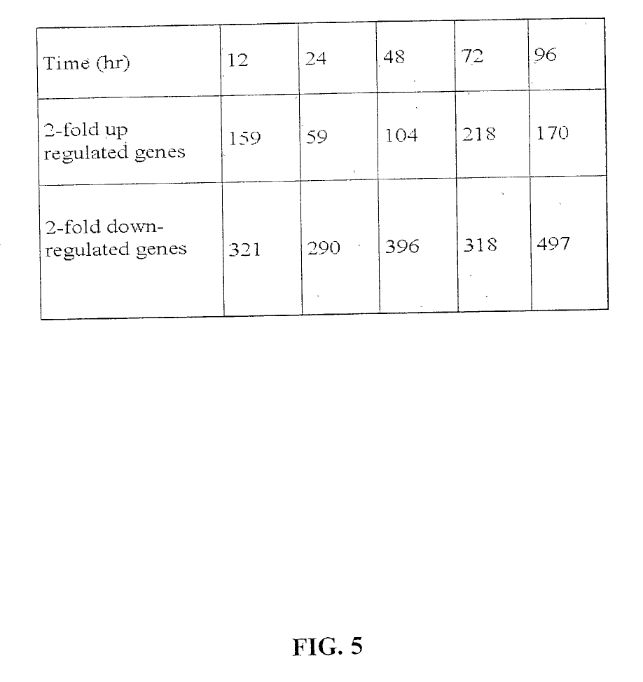

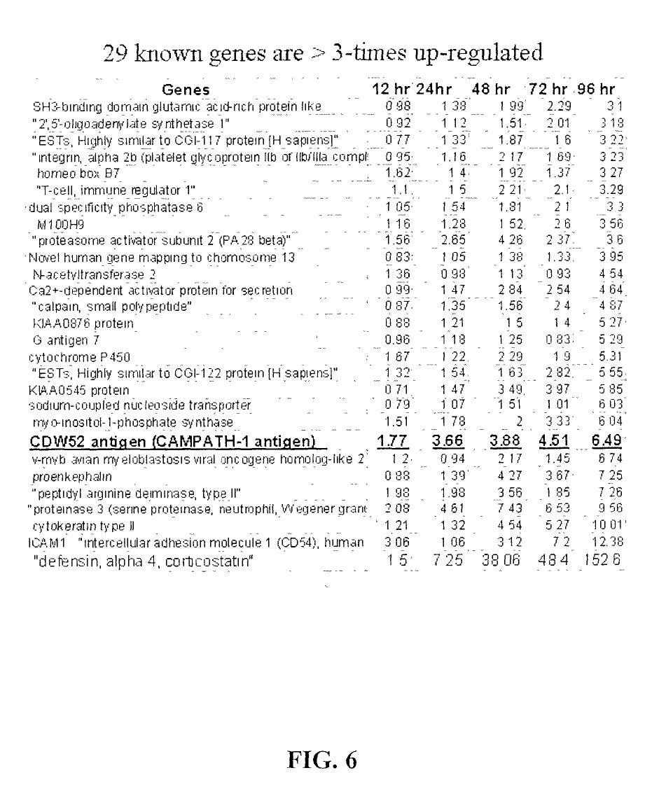

Results of this analysis showed that many genes were up-regulated or down-regulated in ATRA-treated NB4 cells compared to untreated controls. FIG. 5 shows a summary of the number of genes showing two-fold changes in gene expression at various time points. The up- or down-regulated genes varied with time of incubation with ATRA. Both known and unknown genes were included among the ATRA-modulated genes. FIG. 6 shows a list of 29 known genes that displayed at least 3-fold up-regulation following 96-hour exposure of the cells to ATRA. Most of the up-regulated genes were genes known to be related to cell differentiation or neutrophil function, including defensin (alpha 4), proteinase 3, and intercellular adhesion molecule (ICAM1). These genes showed a consistent linear increase with time in level of gene expression, demonstrating the reliability of the cDNA microarray technique.

As shown in FIG. 6, ATRA treatment significantly increased expression of CD52 in the NB4 cells. CD52 mRNA expression increased steadily with increasing time of treatment with ATRA, peaking at 6.49 fold over control after 96 hr.

Consistent with the documented differentiation of the NB4 cells, the gene expression analysis revealed that genes with a known relationship to cell proliferation were down-regulated. These genes included the CCAAT enhancer binding protein, cyclin-dependent kinases, topoisomerase III, and zinc finger proteins.

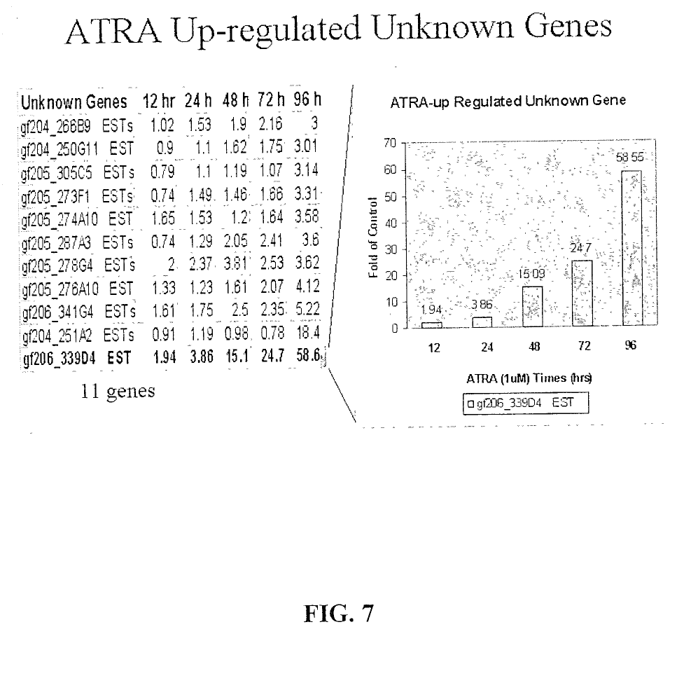

FIG. 7 shows a list of unknown genes (ESTs) that were up-regulated at least 3-fold by ATRA at 96 hrs. As shown on the right of FIG. 7, particularly robust up-regulation was associated with an EST identified as g1206_{—}339D4.

### Example 3

### CD52 Upregulation in ATRA-Treated APL Cells

Reverse transcriptase (RT)-PCR. NB4 cells were cultured and treated with or without ATRA as described above. Some assays were performed using other cell lines including the erythroblastic leukemia cell line K562, the monocytic leukemia cell line U937 and the HL-60 cell line, a human myeloblastic leukemia cell line lacking the t(15; 17) translocation. Total RNA was isolated from the cells and used for CD52 cDNA synthesis by RT-PCR. The cDNA was synthesized in a 20 μl reaction mixture containing 5 μg RNA, 5 mM dNTP, 0.1 μg random primers and 200 U reverse transcriptase, and the mixture was incubated at 42° C. for 1 hr. The cDNA was amplified for 30 cycles with denaturation at 94° C. for 30 seconds, annealing at 56° C. for 30 seconds and extension at 72° C. for 1 min. Amplified products were sequenced and confirmed by comparison with sequence data from GenBank.

Western blot analysis. Cell lysates from control and ATRA-treated NB4 cells were separated on a 4%-20% gradient SDS-polyacrylamide gel and then transferred to PVDF membrane (Millipore, Bedford, Mass.) using a semidry electroblot chamber. The membrane was blocked overnight with 5% dry-milk in Tris-buffered saline and then incubated with anti-CD52 antibody (Serotec, Inc., Raleigh, N.C.) for 1 hour at room temperature. The binding of primary antibody was detected by using peroxidase-coupled second antibody (Sigma). Bound peroxidase was detected by the Super Signal Substrate (Pierce, Rockford, Ill.).

Flow cytometry. APL cells were treated as described above with 1 μM of ATRA for 24, 48, 72, and 96 hrs, and cell differentiation was evaluated by changes in cell morphology, CD11B and CD18 expression, and the NBT reduction test. ATRA treated cells (1×10⁶) were stained with CD18-FITC (Becton Dickinson Biosciences San Jose, Calif.), CD11B-PE, and CD52-FITC antibodies (both from Serotec) following manufacturer's instructions. Multicolor analysis of cell surface CD52, CD18, and CD11B molecules was performed by a FACScan flow cytometer and data were analyzed with CellQuest software (Becton Dickinson Biosciences). Controls consisted of FITC- and PE-conjugated isotype-matched immunoglobulin. Samples were analyzed in triplicate. For every sample, 3×10⁴ cells were analyzed.

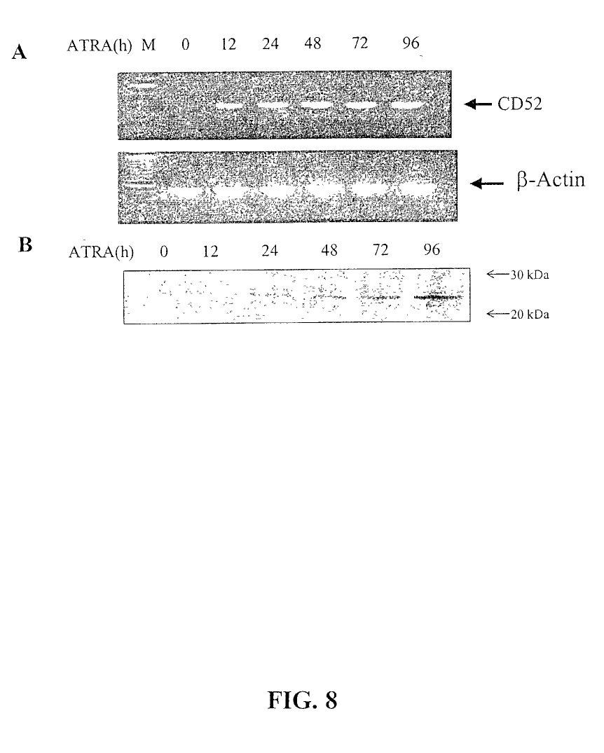

To confirm the results obtained from the microarray studies, the kinetics of CD52 gene expression were studied using RT-PCR assays. As seen in FIG. 8A, expression of CD52 mRNA was not detectable in untreated NB4 cells, but expression was rapidly induced in ATRA-treated NB4 cells. The level of expression of CD52 message increased in proportion to the time of ATRA exposure (12-96 hr). Analysis of levels of β-actin transcripts confirmed equal loading of PCR product in each lane (FIG. 8A, lower lane). Western blot analysis of CD52 protein expression further supported the direct relationship between ATRA treatment of APL cells and CD52 expression. Referring to FIG. 8B, it is seen that multiple protein bands of 21-28 kDa, which are characteristic of CD52 protein (Dyer M J, Semin.Oncol. 26: 52-57, 1999) were only detected in ATRA-treated cells.

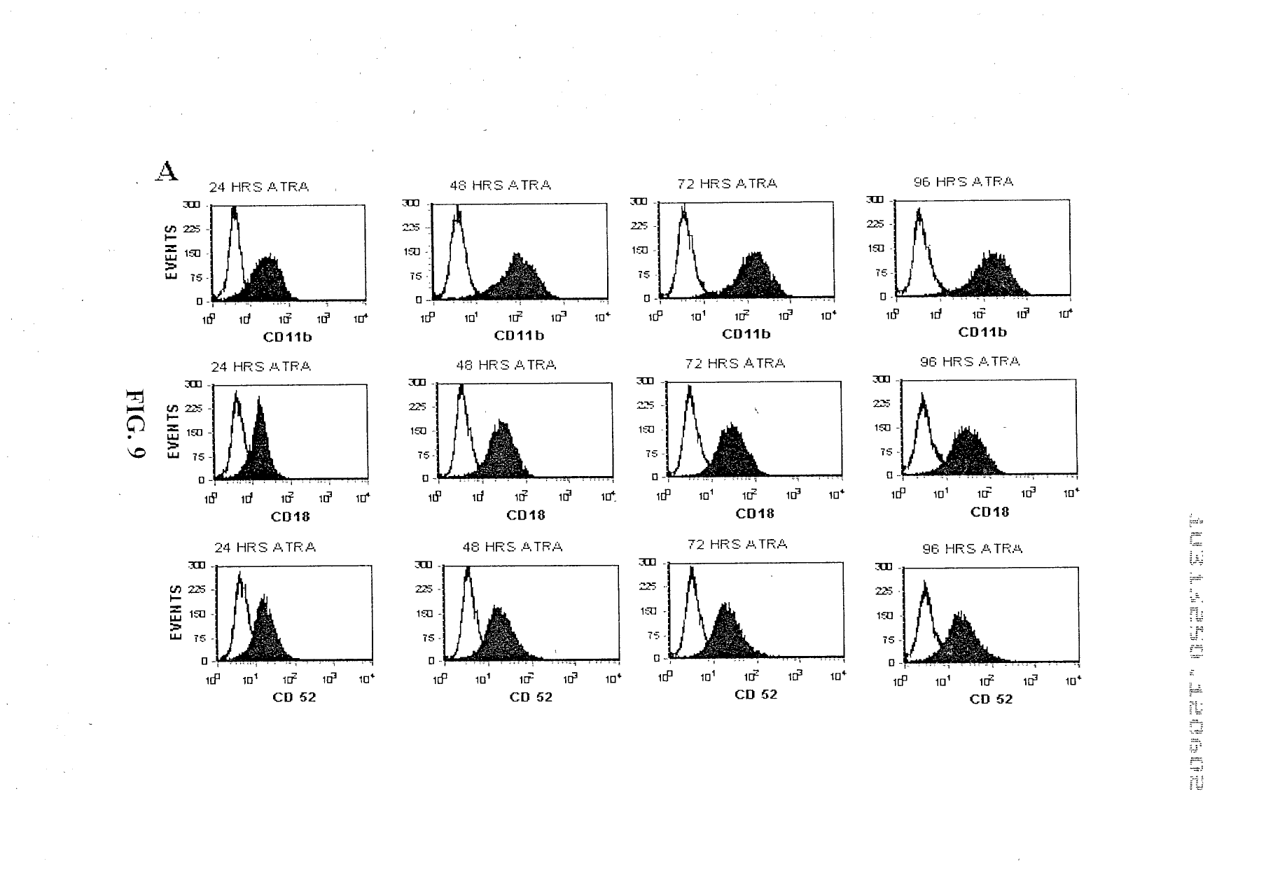

As a further confirmation of the presence of the CD52 molecule on the cell surface following induction by ATRA, induction of CD52 was analyzed using flow cytometry and compared with that of CD11B and CD18. Results of this experiment are shown in FIG. 9. Untreated NB4 cells did not express CD52 on the cell surface. After treatment with ATRA, CD52 protein was expressed in a time-dependent manner on the surface of the cells. CD52 appeared at 24 hr, and reached a maximal expression (˜90%) from 48 to 96 hr of ATRA treatment. Concurrently, CD11B and CD18, markers of granulocytic differentiation (Gahmberg C G, Curr. Opin. Cell Biol. 9: 643-650, 1997), were also upregulated on the ATRA-exposed cells (FIG. 9). Analysis of bivariate plots showed that although at 96 hr of ATRA exposure approximately 80% of the cell population co-expressed CD11B and CD52, approximately 5% of the cells expressed only CD52 while approximately 10% cells expressed only CD11B (FIG. 9).

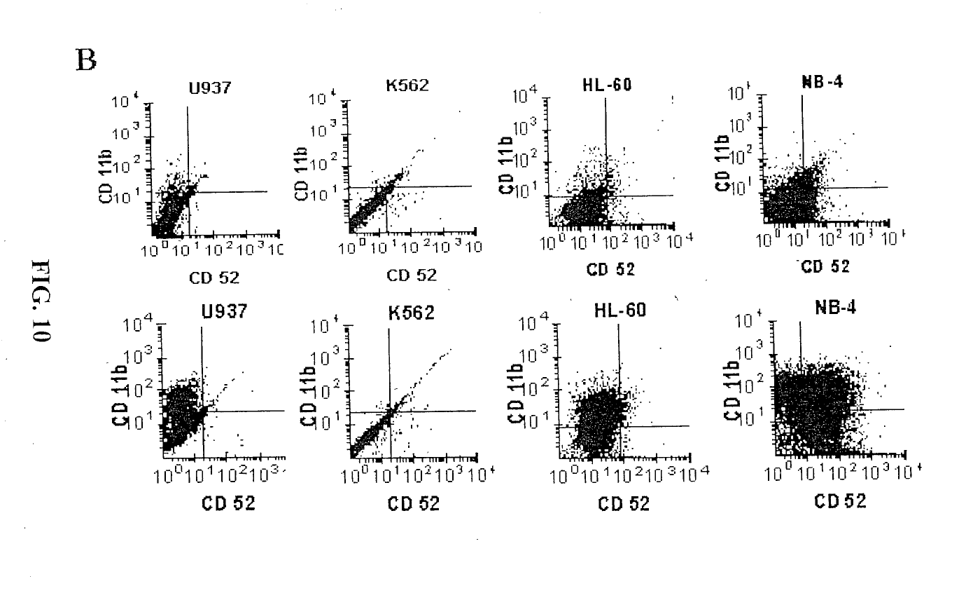

Cell specificity of CD52 expression induction by ATRA. To determine the cell specificity of ATRA-induced CD52 expression, other leukemia cell lines were tested and compared with NB4 under assay conditions used for the NB4 cells. In the monocytic leukemia cell line U937, ATRA slightly increased the expression of CD11B, but did not affect that of CD52 (FIG. 10, upper and lower panels on the left). In the erythroblastic leukemia line K562, ATRA did not induce either CD52 or CD11B expression (FIG. 10, second panels from left). Similarly, CD52 was not up-regulated in ATRA-treated HL-60 cells, human myeloblastic leukemia cells lacking the t(15; 17) translocation. By contrast, robust up-regulation of both CD11B and CD52 was observed in the ATRA-treated NB4 cells (FIG. 10, right panels).

### Other Embodiments

It is to be understood that while the invention has been described in conjunction with the detailed description thereof, the foregoing description is intended to illustrate and not limit the scope of the invention, which is defined by the scope of the appended claim. Other aspects, advantages, and modifications are within the scope of the following claims.

What is claimed is:

1. A method for targeting a molecule to a leukemia cell, the method comprising the steps of:
(a) first contacting the cell with a retinoid in an amount effective to increase the expression of a marker in the cell; and
(b) second contacting the cell with an agent that specifically binds the marker, the agent being selected from the group consisting of the molecule and a substance to which the molecule is attached.

2. The method of claim 1, wherein the marker is selected from group consisting of SH3-binding domain glutamic acid rich like protein, 2′,5′-oligoadenylate synthetase 1, EST highly similar to CGI-117 protein (H. Sapiens), integrin alpha 2b, homeobox B7, T-cell immune regulator 1, dual specificity phosphatase 6, M100HG, PA28 beta, novel human gene mapping to chromosome 13, N-acetyltransferase 2, Ca²⁺-dependent activator protein for secretion, calpain small polypeptide, KIAA0876 protein, G antigen 7, cytochrome P450, EST highly similar to CG1-122 protein (H. Sapiens), KIAA0545 protein, sodium-coupled nucleoside transporter, myo-inositol-1-phosphate synthase, CD52, v-myb avian myeloblastosis viral oncogene homolog-like 2, proenkephalin, peptidyl arginine deiminase type II, proteinase 3, cytokeratin type II, ICAM1, defensin, EST gf²⁰⁴_{—}266B⁹, EST gf204_{—}250G11, EST gf205_{—}305C5, EST gf205_{—}273F1, EST gf205_{—}274A10, EST gf205_{—}287A3, EST gf205_{—}278G4, EST gf205_{—}276A10, EST gf206_{—}341G4, EST gf204_{—}251A2, and EST gf206_{—}339D4.

3. The method of claim 2, wherein the marker is CD52.

4. The method of claim 1, wherein the agent is an antibody.

5. The method of claim 1, wherein the agent is conjugated to a cytotoxin.

6. The method of claim 1, wherein the leukemia cell is a myeloid leukemia cell.

7. The method of claim 1, wherein the leukemia cell is a promyelocytic leukemia cell.

8. The method of claim 1 wherein the leukemia cell is an acute promyclocytic leukemia cell.

9. The method of claim 1, wherein the retinoid is ATRA.

10. A kit for targeting a molecule to a leukemia cell, the kit comprising:
(a) a retinoid for increasing the expression of a marker in the cell; and
(b) an agent that specifically binds the marker.

11. The kit of claim 10, wherein the leukemia cell is selected from the group consisting of a myeloid leukemia cell, a promyelocytic leukemia cell and an acute promyclocytic leukemia cell.

12. The kit of claim 10, wherein the retinoid is ATRA.

13. The kit of claim 10, wherein the marker is the marker is selected from the group consisting of SH3-binding domain glutamic acid rich like protein, 2′,5′-oligoadenylate synthetase 1, EST highly similar to CGI-117 protein (H. Sapiens), integrin alpha 2b, homeobox B7, T-cell immune regulator 1, dual specificity phosphatase 6, M100HG, PA28 beta, novel human gene mapping to chromosome 13, N-acetyltransferase 2, Ca²⁺-dependent activator protein for secretion, calpain small polypeptide, KIAA0876 protein, G antigen 7, cytochrome P450, EST highly similar to CG1-122 protein (H. Sapiens), KIAA0545 protein, sodium-coupled nucleoside transporter, myo-inositol-1-phosphate synthase, CD52, v-myb avian myeloblastosis viral oncogene homolog-like 2, proenkephalin, peptidyl arginine deiminase type II, proteinase 3, cytokeratin type II, ICAM1, defensin, EST gf204_{—}266B9, EST gf204_{—}250G11, EST gf205_{—}305C5, EST gf205_{—}273F1, EST gf205_{—}274A10, EST gf205_{—}287A3, EST gf205_{—}278G4, EST gf205_{—}276A10, EST gf206_{—}341G4, EST gf204_{—}251A2, and EST gf206_{—}339D4.

14. The kit of claim 10, wherein the agent is an antibody that specifically binds CD52.
